(19) United States
(12) Patent Application Publication
(10) Pub. No.: US 2025/0311705 A1
(43) Pub. Date: Oct. 9, 2025

(54) METHOD OF GENETIC MANIPULATION OF NEURAL CELLS IN GYRENCEPHALIC FETAL BRAIN AND USES THEREOF

(71) Applicant: Children's National Medical Center, Washington, DC (US)

(72) Inventors: Nobuyuki Ishibashi, Bethesda, MD (US); Jaya Devina Prasad, Washington, DC (US); Tarik Haydar, Marblehead, MA (US); Zhen Li, Hyattsville, MD (US)

(73) Assignee: Children's National Medical Center, Washington, DC (US)

(21) Appl. No.: 19/097,802

(22) Filed: Apr. 1, 2025

Related U.S. Application Data
(60) Provisional application No. 63/573,985, filed on Apr. 3, 2024.

(51) Int. Cl. A01K 67/0276; C12N 15/85; C12N 15/87

(52) U.S. Cl. CPC A01K 67/0276; C12N 15/8509; C12N 15/87; A01K 2217/054; A01K 2227/108; A01K 2267/0318; C12N 2015/8536

(57) ABSTRACT

The present invention is directed to genetic manipulation of neural cells in the gyrencephalic fetal brain for developing treatment strategies for various neurodevelopmental disorders.

### STATEMENT REGARDING FEDERALLY SPONSORED RESEARCH OR DEVELOPMENT

This invention was made with government support under NS127051 awarded by the National Institute of Health. The government has certain rights in the invention.

### FIELD OF THE INVENTION

The present invention pertains to the fields of genetic manipulation of neural cells in the gyrencephalic fetal brain for developing treatment strategies for various neurodevelopmental disorders.

### BACKGROUND OF THE INVENTION

Diverse networks of neurons and glia produce the advanced computational power of the mammalian brain. Neural precursor cell (NPC) populations present in the ventricular and subventricular zones (VZ and SVZ) of the fetal brain generate all neurons and glia either directly, or indirectly via intermediate progenitors. While there has been a rapid increase in understanding the cell diversity and underlying genetic mechanisms of these precursor cells, most of this work has been accomplished in the lissencephalic rodent (primarily mouse). In fact, the developmental mechanisms for fate specification of most cell populations in the gyrencephalic brain including humans are largely unknown, primarily because there are limited models for this type of exploration. Establishment of a system in which a large gyrencephalic brain can be studied using modern genetic and cellular imaging techniques would significantly impact our understanding of normal human brain development and provide a critical tool for elucidating the etiology and treatment strategies for various neurodevelopmental disorders.

### BRIEF SUMMARY OF THE INVENTION

The present application provides a spatial and temporal characterization of neural precursor cells in the developing Göttingen minipig brain. One aspect of this disclosure is directed to a new animal model and corresponding methods of use for detection, evaluation, or investigation of the effects of different genes on fetal brain development or on brain regeneration or repair. This model uses a large mammal—fetal swine—instead of smaller mammals like mice or rats to model the effects of gene expression in the brain. This model is directed to production and use of a non-naturally-occurring non-human mammal that has been genetically modified to express or not to express particular genes. The model has substantial utility because it is useful for detection, evaluation, investigation or modeling of human brain diseases, disorders, or conditions. Due to use of large fetal mammals, it provides a specific, substantial and credible utility compared to prior murine models. More useful and accurate modeling of the effects of gene expression than prior murine models.

Embodiments of the invention include but are not limited to the following.
1. A genetically modified large fetal mammal, wherein the fetal mammal has been modified to express, enhance, attenuate, or not express at least one gene associated with a brain disease, neurological development, disorder, or condition, and wherein the fetal mammal exhibits symptoms of the brain disease, disorder or condition that are more representative of human disease progression than prior mouse models.
2. The genetically modified large fetal mammal of embodiment 1, wherein the genetically modified large fetal mammal is Sus scrofa domesticus or another member of the Suidae family or Sus genus.
3. The genetically modified large fetal mammal of embodiment 2 that is Sus scrofa, wherein the age of the fetus is 14, 15, 16, 17, 18, 19, or 20 weeks.
4. The genetically modified large fetal mammal of embodiment 2 that is Sus scrofa that has a brain which has undergone, or is undergoing, cortical folding or gyrification. In some alternative embodiments, a neonatal Sus scrofa no more than 1, 2, 3, 4, 5, 6, 7, 8, 9, 10, 11 or 12 weeks of age is used instead of a fetal Sus scrofa.
5. The genetically modified large fetal mammal of embodiment 1, wherein the at least one gene comprises a member of the human NOTCH2NL, ARHGAP11B or NeuroD family, or a Sus scrofa homolog thereof having at least 70, 75, 80, 85, 90, 95, 99, <100, of 100% sequence identity thereto, as described by or incorporated by reference to GENBANK (<https://www.ncbi.nlm.nih.gov/genbank/>), NCBI RefSeq <(https://www.ncbi.nlm.nih.gov/refseq/>), or UniProtKB (<https://www.uniprot.org>/) or to CELL, Fiddes et al.: Human-specific NOTCH2NL genes affect Notch signaling and cortical neurogenesis” www.cell.com/cell/fulltext/S0092-8674(18)30383-0 DOI: 10.1016/j.cell.2018.03.051; CELL, Suzuki et al.: “Human-specific NOTCH2NL genes expand cortical neurogenesis through Delta/Notch regulation.” www.cell.com/cell/fulltext/S0092-8674(18)30399-4 DOI: 10.1016/j.cell.2018.03.067. A representative human NOTCH2NL is described by NCBI Reference Sequence: NM_203458.6; a representative human ARHGAP11B sequence is described by NM_001039841.3→NP_001034930.1, and representative human NeuroDI sequences are described by NM_002500.5→NP_002491.3. In case of ambiguity, the version of a sequence accession number or sequence reference number prior to, but closest to the filing date of the present application, is referenced.
6. The genetically modified large fetal mammal of embodiment 1, wherein the at least one gene comprises human LIS1, ARX, DCX, RELN, TUBA1A, CBLN2, SOX2, EOMES, OLIG2, FOXO1, and/or EGR1, or a Sus scrofa homolog thereof having at least 70, 75, 80, 85, 90, 95, 99, <100, of 100% sequence identity thereto, as described by or incorporated by reference to GENBANK, NCBI RefSeq, or UniProtKB (<https://www.uniprot.org>/).
7. The genetically modified large fetal mammal of embodiment 1 that has been modified to express a human gene or to increase expression of a Sus gene that is a homolog or ortholog of a human gene.
8. The genetically modified large fetal mammal of embodiment 1 that has been transformed by a vector expressing a human gene or to stabilize or increase expression of a Sus gene that is a homolog or ortholog of a human gene.
9. The genetically modified large fetal mammal of embodiment 1 that has been modified to attenuate or knock out expression of a human gene or a Sus gene analogous to a human gene.
10. The genetically modified large fetal mammal of embodiment 1 that has been transformed by a polynucleotide that knocks out expression of a Sus gene analogous to a human gene.
11. A method for modeling therapeutic effects of DNA injection into a minipig fetal brain, comprising:
   electroporating in utero a target polynucleotide into the brain of a fetal minipig in utero,
   imaging the brain structures or expression of the target polynucleotide detect and quantifying changes in brain development or function resulting from the administered polynucleotide vector, and
   comparing the detected changes to a control or control value to assess effects of expression of the target gene in the fetal brain.
12. The method of embodiment 12, wherein a needle containing at least one episomal or integration-based plasmid is inserted into the lateral ventricle to deliver the target polynucleotide.
13. The method of embodiment 12, wherein electroporation is performed under high resolution ultrasound imaging.
14. The method of embodiment 12, wherein the target polynucleotide is tagged with a fluorescent dye or other detectable agent.
15. The method of embodiment 12, wherein the imaging is performed non-invasively.
16. The method of embodiment 12, wherein the imaging is performed ex vivo after removal and/or sectioning of the fetal brain.
17. The method of embodiment 12, wherein said imaging is performed in utero.
18. The method of embodiment 12, wherein said imaging is performed post-mortem ex vivo.
19. The method of embodiment 12, wherein said imaging comprises evaluation how NPCs give rise to apical radial glial cells, (ii) apical intermediate progenitors, (iii) basal intermediate progenitors and/or (iv) basal radial glia, all of which generate neurons that migrate along radial glial fibers to form the six layers of the cortex in an inside-out fashion; or comprises fate mapping of structures in the developing brain.
20. The method of embodiment 12, further comprising diagnosing or treating a human subject after evaluating the comparative changes in brain structure induced by electroporation of the target polynucleotide and/or administering a target polynucleotide or other drug or biologic to the human subject.
21. The method of claim 12, further comprising diagnosing a human subject, who may be a neonate or a fetus, after evaluating the comparative changes in brain structure induced by electroporation of the target polynucleotide and/or conducting MRI or ultrasound visualization of the brain of the human fetus or neonate.
22. The method of claim 12, further comprising treating a human subject, who may be a neonate or a fetus, after evaluating the comparative changes in brain structure of the fetal minipig induced by electroporation of the target polynucleotide and/or administering a drug, biologic or other treatment.

A more complete appreciation of the disclosure and many of the attendant advantages thereof will be readily obtained as the same becomes better understood by reference to the following detailed description when considered in connection with the accompanying drawings below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete appreciation of the invention and many of the attendant advantages thereof will be readily obtained as the same becomes better understood by reference to the following detailed description when considered in connection with the accompanying drawings, wherein:

FIGS. 1A-1C. Experimental design of the fetal in utero electroporation surgery. (FIG. 1A) under anesthesia, the uterus of the time-mated pregnant Göttingen minipigs was partially exteriorize. (FIG. 1B) An ultrasound system was used to provide real-time images of the guided injections of plasmids of interest into the lateral ventricle of the fetal minipig brain. (FIG. 1C) Following the surgery, the pregnant minipig was recovered to a range of time-points, where it was then euthanized, the fetal minipigs were collected and the brains extracted and analyzed for various outcome measures. Scale bar=10 mm.

FIGS. 2A-2D. Representative examples of the developmental trajectories of minipig brain development from G7.5W to adulthood (387 days). Scale bar=10 mm. NB. The brain undergoes extensive growth between G7.5W (FIG. A) and G10.5W (FIG. 2B), in which the primary gyri, which can be observed in the adult brain (FIG. 2D), appear to be established between G8.5W (not shown) and G10.5W. In addition, over a one-week period, increased development and complexity of the primary gyri can be observed between G10.5W and G11.5W (FIG. 2C).

FIG. 3. Schematic of fetal brain development from ˜G7.5W (left), G8.5W (middle), to G11.5W (right) in the fetal minipig depicting the proposed points of expansion resulting in the formation of the primary gyri, which can be observed in the adult minipig brain.

FIG. 4A-4C. Validating the feasibility of performing in utero electroporation in the fetal minipig as an approach for labelling cells of interest in order to study NPC dynamics in the fetal minipig brain. (FIG. 4A) Validation of the IUE protocol in the fetal minipig where co-electroporation of hyperactive version of piggyBac transposase (hyPBase) from CAG promoter and LiOn integration-coupled translational switch, expressing the red fluorescent protein (RFP) from a CAG promoter. CAG-RFP sequence is integrated into mammalian genome for long term fluorescent protein expression. (FIG. 4B) Validation of short inhibitory RNA (siRNA) vectors targeting EOMES cells (Tbr2), where the siRNA works by silencing the Tbr2 gene. (FIG. 4C) Validation of (i) integration-based plasmid using the hyperactive version of piggyBac transposase (hyPBase) targeting the NueroD4 population of cells, and (ii) longer-term detection of fluorescence.

FIGS. 5A-5C. Vector maps for pNeuroD4-hyPBase (Upper, FIG. 5A), LiOn*-CAG∞RFP (middle, FIG. 5B), and Pcglh-siEOMES-1 (lower, FIG. 5C) plasmids. The pNeuroD4-hyPBase is generated in-house using the Pcag-hyPBase by swapping the CAG promoter with the mouse NeuroD4 promoter. LiOn*-CAG∞RFP is a gift from Jean Livet (Addgene plasmid #154019). The Pcglh-siEOMES-1 plasmid is made from Pcglh-luciferase, where the siRNA against luciferase is replaced with one against mouse Eomes. Pcglh-luciferase is a gift from Angelique Bordey (Addgene plasmid #194979).

FIGS. 6A-6B. Validating tissue mapping of NPCs in the developing brain. FIG. 6A: Overview of the cyclic multiplexing protocol used to map NPCs in the developing minipig brain. Adapted from Maric et al., 2021. FIG. 6B: Antibody validation for markers used for mapping NPC, as well as cortical layer markers of interest. Antibodies used include, respectively, Paired-box 6 (Pax6); Vimentin (VIM); SRY-box 2 (Sox2); Doublecortin (DCX); EOMES cells (Tbr2); Glial fibrillary acidic protein (GFAP); Special AT-rich sequence-binding protein 2 (SATB2); Neurogranin (NRGN); B cell leukemia/lymphoma 11B (CTIP2); Forkhead box protein P2 (FOXP2). This technique allows for the direct comparison of multiple markers, all on a single tissue.

FIGS. 7A-7D. FIG. 7A: Overview of cyclic multiplex immunohistochemistry labelling of NPCs in the fetal minipig brain at ˜G7.5W and schematic (FIG. 7C) of a minipig brain at G8.5W where the white dotted box “B” (FIG. 7A) and red dotted box “D” in FIG. 7C depict magnified regions of interest. Magnified region marked by the dotted box (FIGS. 7B, 7D) illustrating the organization of Neuroblasts (FIGS. 7B.i, 7D.i), NPCs (FIGS. 7B.ii, v, vi, and 7D.ii, 7Dv, vi), basal intermediate progenitor cells (FIGS. 7B.iii, 7D.iii), and astrocytes (FIGS. 7B.iv, 7D.iv). Minimizing blood vessel autofluorescence requires further optimization, as is particularly evident in FIGS. 7B.ii, 7B.iv, 7D.ii, 7D.iii and 7D.iv.

FIGS. 8A-8C. Under anesthesia, a vertical incision was performed mid abdomen, first through the skin and underlying abdominal muscle to expose the uterus (FIG. 8A). Guided by the high resolution ultrasound imaging system, a sterilized needle with microliter syringe (Hamilton Company) containing plasmid (episomal or integrating plasmids) was visualized in real time and inserted through the maternal uterus and fetus's skull, directly into the lateral ventricle of the fetal brain (FIG. 8B). As shown, experiments targeted the cerebral cortex with episomal expression plasmids, which were allowed to express for 24 hours (FIG. 8C) and provide examples of successful gene manipulation using a variety of plasmids and across multiple time-points.

### DETAILED DESCRIPTION OF THE INVENTION

The developing brain undergoes profound changes during the first two decades of life, involving a series of intricately orchestrated cellular, morphological, and biochemical neurodevelopmental events. Understanding the mechanisms underlying these events is critical to furthering our understanding of human brain development, and the disorders that affect human brain development. The neocortex develops from a diverse pool of neural precursor cells (NPCs) that produce all of the neurons and glial cells necessary for the precise formation and refinement of brain networks that underpin all human physiological functions. Proliferation of these NPCs gives rise to at least three unique groups of intermediate progenitors: (i) apical intermediate progenitor cells in the ventricular zone (VZ), (ii) basal intermediate progenitor cells and (iii) basal radial glia, both of which reside in the subventricular zone (SVZ). Together, these NPCs generate neurons that migrate along radial glial fibers to form the six layers of the cortex in an inside-out fashion. However, it is unclear how these precursors produce the full spectrum of mature neuronal classes.

Piglets are a powerful model with which to study complex brain development because they have a highly evolved gyrencephalic neocortex and developmental processes of gyrifications similar with monkey, ape and human brain. We have established for the first time an in utero electroporation (IUE) approach in the fetal minipig (Sus scrofa dom.) which allows efficient genetic manipulation of specific cell types to track self-renewing and neuron-generating divisions that form gyri during the fetal period. Establishment of this technique in the minipig model can be used to define the precise time, birthplace and lineage of young migrating neurons, elucidate the key steps and cell types required for gyrencephaly, and the mechanisms underlying the maturation and integration of neurons in the gyrencephalic neocortex. In addition, this experimental system will dramatically advance our ability to model a wide range of neurodevelopmental diseases and facilitate development of novel therapeutic approach in the future.

Swine include members of the family Suis as well as the genus Sus. A preferred type of fetal swine are minipigs as exemplified herein. Minipigs or miniature pigs that may be used include the Göttingen minipig as exemplified herein which is a breed of miniature pig known for its exceptionally small size, docile nature, and well-characterized health status. Other miniature pigs which may be used include the Vietnamese Pot-Bellied Pig, Juliana Pig, KuneKune Pig, Ohmini Pig, Yucatan Miniature Pig, Czech Minipig and Hanford Miniature Pig.

Genes involved in brain development. One notable family of genes involved in brain development is the NOTCH2NL family. These genes are human-specific and play a crucial role in the expansion of the cerebral cortex by delaying the differentiation of cortical stem cells into neurons, thus allowing for a larger pool of stem cells and ultimately more neurons in the neocortex.

Another significant gene involved in brain development is ARHGAP11B, also human-specific. It contributes to the expansion of the neocortex by increasing the pool of basal progenitor cells, which are essential for neocortical development and evolution.

In terms of brain repair, genes like NeuroD1 are important. NeuroD1 is a transcription factor that can convert glial cells into functional neurons, offering a potential approach for neural repair after injuries such as ischemic stroke.

Variants of any of the genes, polynucleotides, or amino acid sequences disclosed herein or incorporated by reference, may contain 1, 2, 3, 4, 5, 6, 7, 8, 9, 10, 11, 12, 13, 14, 15, 16, 17, 18, 19, 20 or more deletions, substitutions, or additions of nucleotides or amino acid residues.

Variants of any of the genes, polynucleotides, or amino acid sequences disclosed herein, including paralogs, engineered sequences, mutants, alternative splice variants, polymorphs, isotypes, or isologs, may have 70, 75, 80, 85, 90, 95, 99, <100, or 100% sequence identity or similarity to any of the polynucleotides or polypeptides disclosed herein. These ranges include all intermediate values and subranges.

BLASTN may be used to identify a polynucleotide sequence having at least 70%, 75%, 80%, 85%, 87.5%, 90%, 92.5%, 95%, 97.5%, 98%, 99% <100% or 100% sequence identity to a reference polynucleotide such as those identified herein by accession or reference number or those specifically incorporated by reference. A representative BLASTN setting modified to find highly similar sequences uses an Expect Threshold of 10 and a Wordsize of 28, max matches in query range of 0, match/mismatch scores of 1/−2, and linear gap cost. Low complexity regions may be filtered or masked. Default settings of a Standard Nucleotide BLAST are described by and incorporated by reference to hypertext transfer protocol secure://blast.ncbi.nlm.nih.gov/Blast.cgi?PROGRAM=blastn&PAGE_TYPE=BlastSearch&LINK_LOC=blasthome (last accessed Mar. 18, 2025).

BLASTP can be used to identify an amino acid sequence having at least 70%, 75%, 80%, 85%, 87.5%, 90%, 92.5%, 95%, 97.5%, 98%, 99% or <100% sequence identity, or similarity to a reference amino acid, using a similarity matrix such as BLOSUM45, BLOSUM62 or BLOSUM80 where BLOSUM45 can be used for closely related sequences, BLOSUM62 for midrange sequences, and BLOSUM80 for more distantly related sequences. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity or similarity to the amino acid sequences disclosed herein are contemplated and encompassed by this disclosure. A representative BLASTP setting that uses an Expect Threshold of 10, a Word Size of 3, BLOSUM 62 as a matrix, and Gap Penalty of 11 (Existence) and 1 (Extension) and a conditional compositional score matrix adjustment. Other default settings for BLASTP are described by and incorporated by reference to the disclosure available at: hypertext transfer protocol secure://blast.ncbi.nlm.nih.gov/Blast.cgi?PROGRAM-blastp&PAGE_TYPE=BlastSearch&LINK_LOC-blasthome (last accessed Mar. 18, 2025.

Amino acid sequences may be deduced from the corresponding polynucleotide sequences using the genetic code and vice versa taking into account the degeneracy of the code.

Vectors and episomes. Those skilled in the art may select an appropriate vector for transduction and expression of a polynucleotide in utero. These include but are not limited to minicircle DNA vector which can promote robust and persistent gene expression; S/MAR-based episomal vectors that provide fore episomal replication and nuclear retention; lentiviral vectors capable of transducing dividing and non-dividing cells; adeno-associated virus vectors which can be engineered with tissue-specific promoters to enhance target polynucleotide or gene expression in specific tissues; and bacteriophage lambda vectors which can be engineered for targeted gene delivery. In some embodiments short inhibitory RNA (siRNA) vectors may be used such as H1 or U6 vectors; see Thermofisher, siRNA Expression Vectors with Selectable Markers For Long Term Studies in Mammalian Cells (siRNA Expression Vectors with Selectable Markers|Thermo Fisher Scientific—US, last accessed Mar. 19, 2025) and Chen, et al. J RNAI GENE SILENCING. 2005 Jul. 27;1(1):5-11 both of which are incorporated by reference.

Dyes for tagging polynucleotides. In some embodiments, DNA or RNA for electroporation into a cell may be tagged with a detectable marker. When tagging DNA for electroporation or injection into an animal, several fluorescent dyes can be used including but not limited to Cy3 and Cy5, Alexa Fluor Dyes and Hoechst Dyes (e.g., Hoechst 33258, Hoechst 33342. Those skilled in the art may select a suitable dye for tagging DNA or target agents in vivo or for

Electroporation in utero. Various modes for performing electroporation may be used in conjunction with the disclosed methods, preferably a pliable Electroporation Patch (ep-Patch) is employed as described in the Examples. This method uses a flexible patch with closely spaced electrodes to deliver DNA or siRNA into tissues non-invasively, providing uniform electric fields and efficient delivery. Alternatively, gene paddles electrodes which are paddle-style electrodes designed for in vivo applications, including muscle and brain tissues may be used. They are suitable for delivering DNA into various tissues with high efficiency. Tweezertrods electrodes may be employed which are similar to pin-and-paddle tweezers and are reusable and non-invasive, offering a different design for localized electroporation. In some embodiments, the electroporation settings for in utero electroporation may be selected as follows: voltage: 100-125 V; waveform type: square; number of pulses: 5, pulse duration: 50 msec and pulse intervals: 1 second. In other embodiments electroporation settings will vary ±1, 2, 5, 10, 20 or 50% of those described above (or any intermediate value of subrange thereof). DNA concentration can vary based on the plasmid volume of 10-15 μl.

In some alternative methods, other physical methods including but not limited to magnetoporation, sonoporation, and optoporation may be used instead of, or in addition to, electroporation. These are non-invasive methods that use magnetic fields, ultrasound, or light to create pores in cell membranes, respectively. They offer alternatives to electroporation with less potential of damaging cells.

In some embodiments integration of an episome or plasmid vector carrying the target polynucleotide may use an integration-coupled On genetic switch, iOn, which triggers gene expression upon incorporation into the host genome through transposition, thus enabling rapid and accurate identification of integration events following transfection with naked plasmids; see Kumamoto, et al., Direct Readout of Neural Stem Cell Transgenesis with an Integration-Coupled Gene Expression Switch, NEURON. 2020 Aug. 19;107(4):617-630.e6. doi: 10.1016/j.neuron.2020.05.038. Epub 2020 Jun. 18. (incorporated by reference).

In another embodiment, the method may comprise, or further comprise, brain tissue mapping as described by, and incorporated by reference to Maric, et al., Whole-brain tissue mapping toolkit using large-scale highly multiplexed immunofluorescence imaging and deep neural networks, Nature Communications volume 12, Article number: 1550 (2021).

Other modes of introducing polynucleotides or target agents into the fetal brain. The target polynucleotides or vectors containing them or other agents for evaluation may be administered into a fetal brain by a variety of routes when electroporation is not used or used in conjunction with non-electrophoretic delivery. Preferably, can be delivered via intrathecal (into the subarachnoid space), intracerebroventricular (into the cerebral ventricles), or intraparenchymal (directly into brain tissue). Other drugs or agents may be administered into other parts of the fetus or into the placenta or uterus or into the mother. Drugs that can cross the blood-brain barrier may be administered orally or parenterally, for example, by oral administration or by intravenous, intramuscular, or subcutaneous administration into the fetus or mother (animal carrying the fetus).

In vivo evaluation of the fetal brain. Developmental or induced changes to a fetal brain may be detected or evaluated non-invasively in vivo by methods known to those skilled in the art. These methods include, but are not limited to, in utero magnetic resonance imaging (iuMRI) which provides detailed images of brain structures and can monitor changes over time, for example, over the gestation period of a minipig fetus. In some embodiments of the MRI-based techniques, contrast agents like gadolinium may be used to enhance visualization of particular neurological structures or changes to them however such agents are generally not required; non-invasive Fetal Electroencephalography (EEG) which measures fetal brain activity directly, offering insights into brain health and potential injury and can provide real-time information on brain activity; and fetal magnetoencephalography (MEG) which is a non-invasive technique that measures the magnetic fields generated by electrical activity in the fetal brain.

Ex vivo post-mortem evaluation of the fetal brain. Various techniques for evaluating and detecting structures in a developing fetal brain are known to those skilled in the art. These include but are not limited to post-Mortem MRI (Magnetic Resonance Imaging) which is a non-invasive technique that allows for the detailed examination of brain anatomy, tissue composition, and structural connectivity; stereotaxic cutting and histological analysis which involves the precise cutting of post-mortem brains into sections using stereotaxic instruments. These sections are then analyzed histologically to examine microscopic brain structures; and post-mortem dissection and histopathological examination which involves dissecting the brain post-mortem and examining slices of brain tissue under a microscope.

Preferably, changes in the fetal brain are evaluated with respect to those in a control brain that has not been electroporated with a polynucleotide vector or episome thus allowing for a detailed comparative microscopic examination of specific brain regions, enabling precise comparisons between control and polynucleotide treated brains.

Additional applications of the disclosed in utero minipig model. Those skilled in the art may employ the disclosed in utero minipig model disclosed herein to investigate how human brain development or treatment can affect neurodevelopmental disorders, such as autism, ADHD or cerebral palsy, including how factors such as maternal health conditions or environmental exposures, or treatments of mother or fetus with particular biologics or drugs influence fetal brain development. By studying these interactions, scientists can gain insights into the origins of neurodevelopmental disorders like autism, ADHD, or cerebral palsy. The disclosed products and methods using fetal minipigs can model phenomena occurring in the human brain during fetal development and lead to discovery of early interventions or preventive measures of human neurodevelopmental disorders.

The disclosed genetically modified fetuses and methods may be used to develop new therapies for brain disorders or injuries, for example, by detecting the regenerative capacity of the human fetal brain and how it can be applied to repair damaged brain tissue in adults or children.

Additionally, the disclosed genetically modified fetuses and methods may be used to develop or refine neuroimaging techniques and monitoring tools. These could help identify potential developmental issues early in pregnancy based on the minipig model, allowing for timely interventions.

### EXAMPLES

### Validation of the Fetal Minipig as Model of Human Brain Development.

The fetal minipig as a large animal model of human brain development was validated as disclosed below in order to morphologically and temporally characterize NPCs in the VZ and SVZ. The following methods were employed. Under anesthesia, time-mated fetal Göttingen minipigs (7-8 weeks of gestation) were partially exteriorized for in utero electroporation. Following the procedure, animals were recovered for 2 days (D), 7D or 30D, and the brains collected for anatomical and cellular analyses.

The inventors' work defines the structure and folding patterns of the fetal minipig brain during critical periods of neurogenesis and elucidates the structural and developmental similarities and differences between the developing minipig brain and the developing human brain, which are be essential for studies uncovering the mechanisms of complex morphometric development of the brain.

### Example 1. The Spatial and Temporal Characterization of Neural Precursor Cells in the Developing Göttingen Minipig Brain

The neocortex develops from a diverse pool of neural precursor cells (NPCs) that produce all the neurons and glial cells required for the precise formation and refinement of brain networks that underpin all human physiological functions. Studies in lissencephalic rodents have illustrated how NPCs give rise to (i) apical radial glial cells, (ii) apical intermediate progenitors, (iii) basal intermediate progenitors and (iv) basal radial glia, all of which generate neurons that migrate along radial glial fibers to form the six layers of the cortex in an inside-out fashion. However, the relationship between NPCs and the complex patterning of development observed in a gyrencephalic brain has been largely unexplored. The minipig presents itself as an excellent model for studying these complex in utero developmental events due to their prolonged gestational period (term; 16 weeks) and gyrencephalic brain. However, due to the lack of technical studies in the fetal minipig, the use of in vivo cell labelling techniques to study NPC fate mapping, as well as the developmental trajectory of fetal minipig brain development is unclear.

### Validation of the Fetal Minipig as a Large Animal Model of Human Brain Development in Order to Characterize NPCs in the VZ and SVZ.

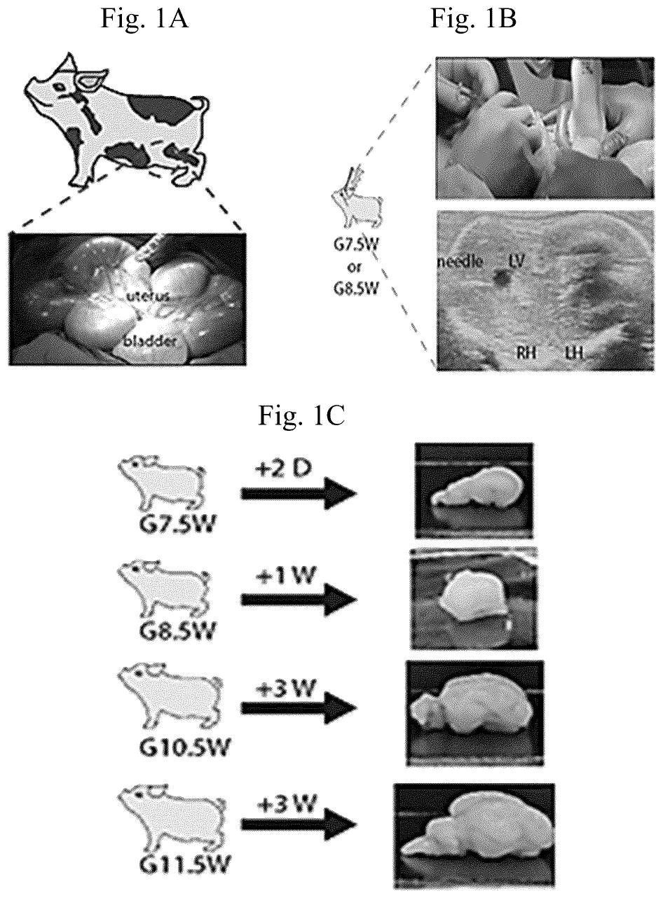

Methodology: Under anaesthesia, the uterus of pregnant Göttingen minipigs (˜7.5-8.5 weeks (W) of gestation (G); n=5) was partially exteriorized (FIG. 1A). Fetal brains were located via ultrasound and were used to guide the needle containing either episomal or integration-based plasmids into the lateral ventricle (FIG. 1B). In utero electroporation (IUE) was performed using a pin-and-paddle tweezers-type electrode and an electroporator. Animals were recovered to G7.5W, G8.5W, G10.5W and G11.5W, and fetal brains collected for analyses (FIG. 1C).

As explained below, in some embodiments, short inhibitory RNA (siRNA) vectors that target EOMES cells (Tbr2) or integration-based plasmids targeting NueroD5 may be used.

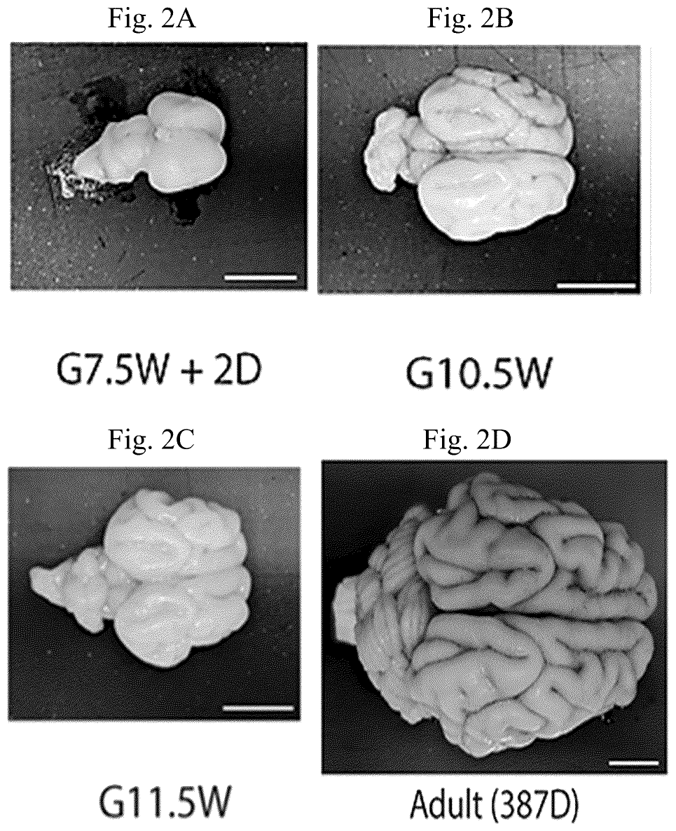

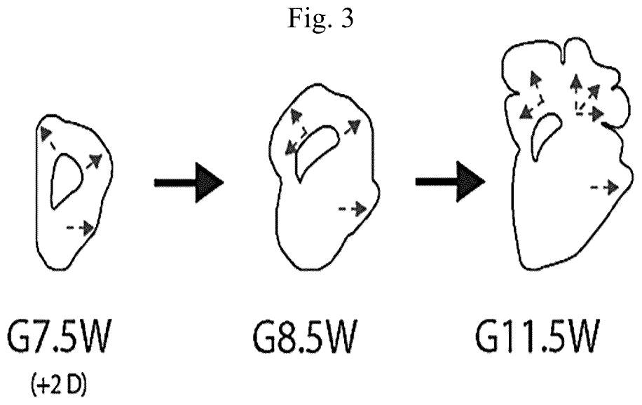

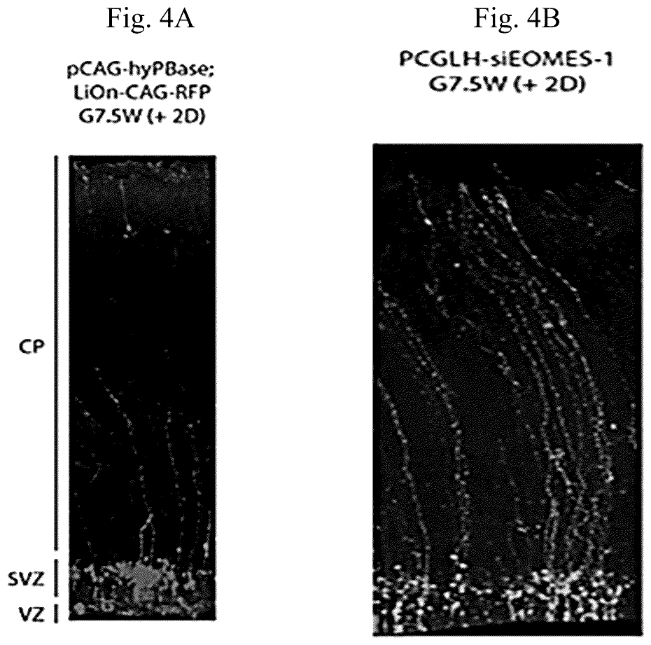

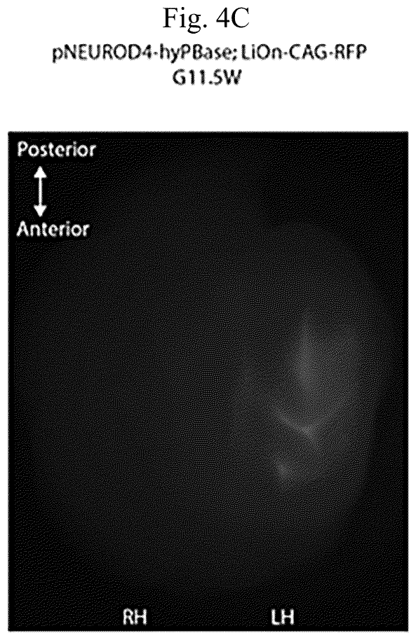

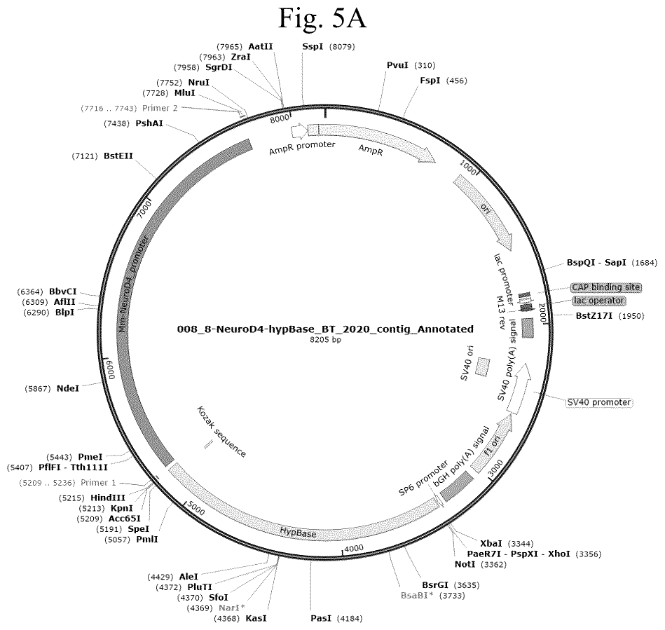

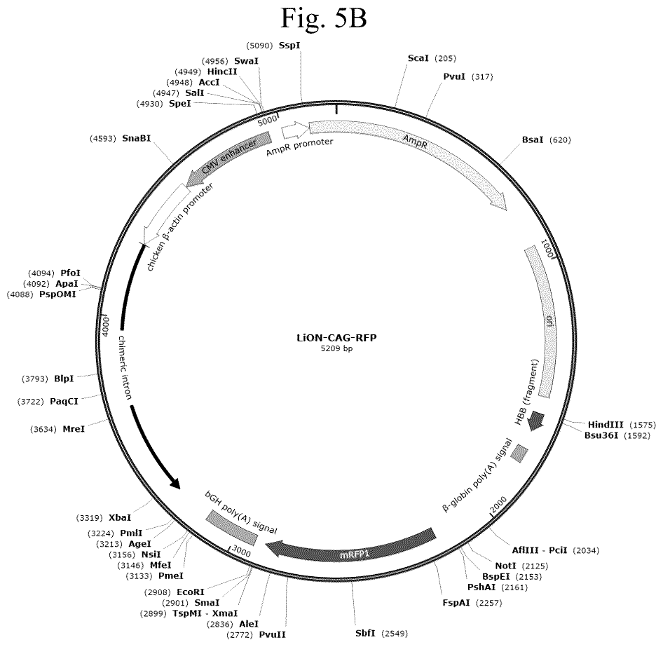

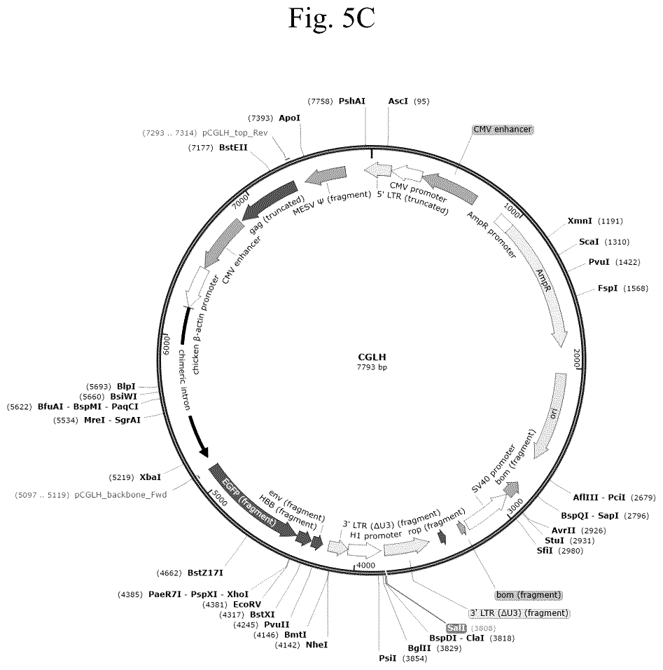

Results: Developmental trajectory of the minipig brain from G7.5W to adulthood (387 days) are shown in FIG. 2. The inventors found that the primary gyri, which can be observed in the adult brain, appear to be established between G8.5W and G10.5W in the fetal minipig brain. FIG. 3 displays schematic of fetal brain development from ˜G7.5W to G11.5W in the fetal minipig depicting the proposed points of expansion resulting in the formation of the primary gyri, which can be observed in the adult minipig brain. These results indicate that fetal IUE is a technically feasible approach for labelling cells of interest in order to study NPC dynamics in the fetal minipig brain (FIG. 4). The inventors successfully validated short inhibitory RNA (siRNA) vectors targeting EOMES cells (Tbr2), integration-based plasmids targeting the NueroD4 population of cells, and longer-term detection of fluorescence (FIG. 4). More details are described in next section (Experimental 2), and the vector map used are shown in FIG. 5. The pCAG-hyPBase plasmid was purchased from VectorBuilder. pNeuroD4-hyPBase was generated in-house using the pCAG-hyPBase by swapping the CAG promoter with the mouse NeuroD4 promoter. LiOn*-CAG∞RFP was a gift from Jean Livet (Addgene plasmid #154019)1. pCGLH-siEOMES-1 plasmid was made from pCGLH-luciferase, where the siRNA against luciferase was replaced with one against mouse Eomes. pCGLH-luciferase was a gift from Angelique Bordey (Addgene plasmid #194979). IUE in the fetal minipig can be successfully performed between G7.5-8.5W, with labelled cells visible up to 3 weeks post IUE.

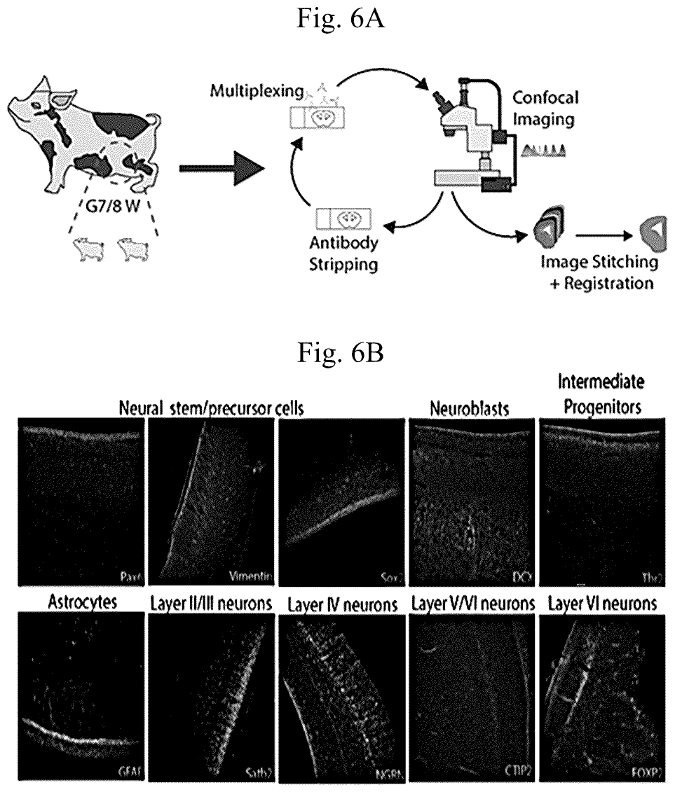

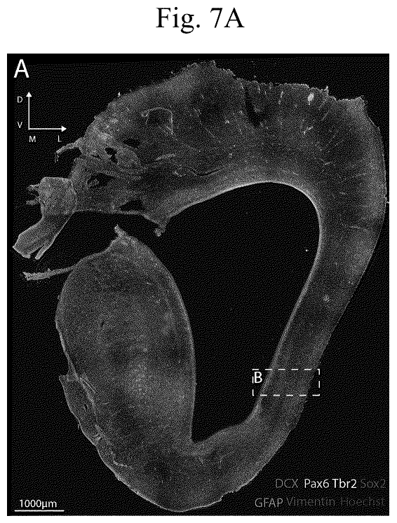

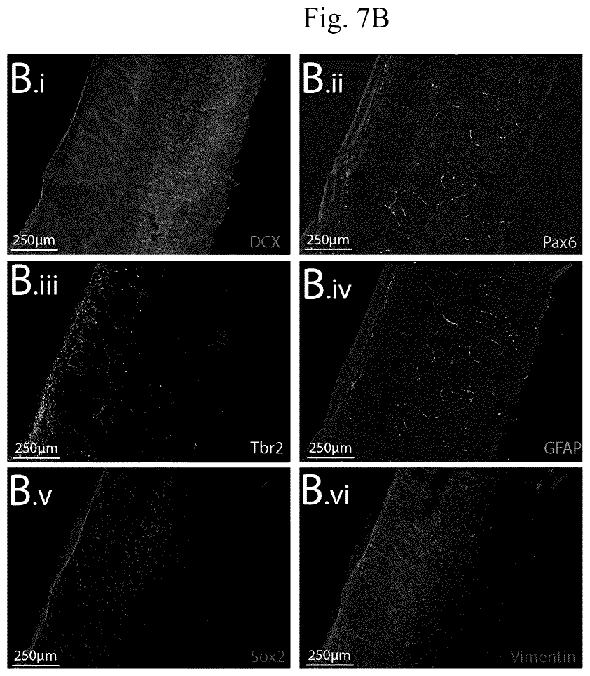

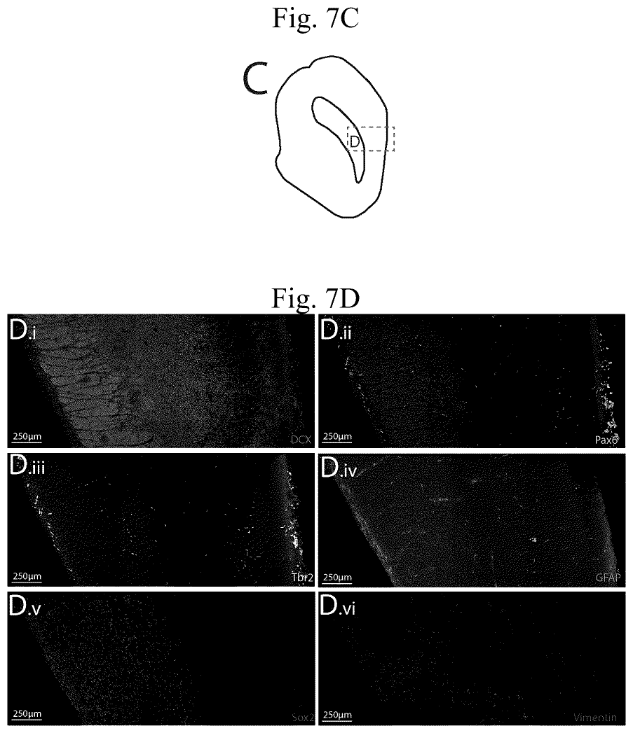

Cyclic multiplexing is a powerful tool² that can be performed in the fetal minipig brain, in order to spatially and temporally map NPCs. Although further optimization is required to reduce tissue autofluorescence, the inventors successfully validated tissue mapping of NPCs in the developing brain as shown in FIG. 6. Overview of cyclic multiplex immunohistochemistry labelling of NPCs in the fetal minipig brain at ˜G7.5W and schematic of a minipig brain at G8.5W are shown in FIG. 7. Most importantly, the fetal minipig brain undergoes a period of profound growth and development between G8.5W and G10.5W. Thus, future studies will focus on combining these techniques in order to elucidate the timeline of gyrus formation, as identifying this timeline will be critical for uncovering the complex mechanisms of morphometric brain development.

### Example 2: In Vivo Genetic Manipulation of Neural Cells in Gyrencephalic Fetal Brain

Fetal development is one of the last frontiers of medical science. It is an exceedingly short time for the rapid biological processes that must occur properly and on schedule to ensure normal function after birth, a period when stem cells differentiate in precise ways to build the complex human body. Unlocking the mechanisms of fetal development will impact all sectors of medicine, not just those pertaining to prenatal events but also those important for aging and regeneration. Unfortunately, advances in human fetal medicine have been stalled by several factors, including ethical considerations, the fragility of the period, and by a dearth of appropriate model systems. In fact, the developmental mechanisms for fate specification of most cell populations in the human brain are largely unknown, primarily because there are limited models for this type of exploration. Establishment of a system in which a large gyrencephalic fetal brain can be studied using modern genetic and cellular imaging techniques would significantly impact our understanding of normal human brain development and provide a critical tool for elucidating the etiology and establishing treatment strategies for various neurodevelopmental and neurodegenerative disorders.

Proper development and function of the human body depends on a complex choreography of cell division, migration, and connectivity. Much of what we know about human brain development comes from postmortem studies and experiments on model organisms, most specifically the mouse. However, substantial differences in brain structure and cognitive function between mouse and human prevent rapid advancement towards important clinically relevant goals, particularly those related to higher cognitive functions and to care during the prenatal period. While other advanced species with complex brains, such as ferret and non-human primate (NHP), are used to study neuroscience and other scientific fields, these models either have altered gestational programs compared to human (ferret) or are exceedingly difficult to use for fetal medicine (NHP). To address this, we developed, for the first time, techniques of in vivo genetic manipulation of neural cells in gyrencephalic fetal brain using the minipig. Due to the similarities to human physiology and anatomy, the pig has long been used for biomedical research, most notably in toxicology, drug discovery, and cardiology studies. In addition to similar physiology, the size of the minipig (both the adult sows and the uterine environment) represents a biomedical “sweet spot” because it is close to humans to enable technical innovations that can directly translate to clinical approaches.

The inventors established surgical and imaging approaches to circumvent the opaque minipig uterus and its contents, using in utero electroporation (IUE) as a way to deliver expression vectors into the fetal minipig brain for ectopic gene expression. The ability to visualize and fixate the fetal brain was made possible by high resolution ultrasound imaging, allowing us to monitor the intraventricular injection in real time. Our innovative technical approaches include not only direct access to the gyrencephalic fetal brain but also weeks-long genetic manipulation of neural precursors using several different types of probes as outlined below. The new in vivo genetic manipulation technique in gyrencephalic fetal brain will generate a new platform for preclinical investigation, the development of surgical tools, therapeutic approaches, and imaging paradigms. The potential impact of this innovation is very high; the level of foundational knowledge and methodological development facilitates well-controlled studies by many research groups and companies, leading to development of the fetal medicine and intervention in areas ranging from intellectual disabilities and congenital anomalies to neurodegeneration and cancer.

Methods: Animals. All experiments were performed in compliance with the guidelines of the National Institutes of Health's “Guide for the Care and Use of Laboratory Animals,” and were approved by the Children's National Medical Center Animal Care and Use Committee. Time-mated pregnant Göttingen minipigs (Marshall BioResources, North Rose, NY, USA) were purchased at ˜6 weeks of gestation and housed in the research animal facility at Children's National Hospital.

Surgical procedures. At ˜7 weeks of gestation, pregnant Göttingen minipigs were sedated with intramuscular ketamine and xylazine, intubated, and ventilated (FiO2, 0.21; 12-15 breaths/min) with a volume control ventilator (Servo ventilator 300, Siemens). Prior to surgery, fentanyl, cefazolin sodium, and heparin (300 IU/kg) were administered through a peripheral intravenous line. Anesthesia was maintained by inhalant isoflurane. Mean and systolic arterial pressure, heart rate, and rectal temperature were monitored continuously throughout each experiment and recorded every 15 minutes.

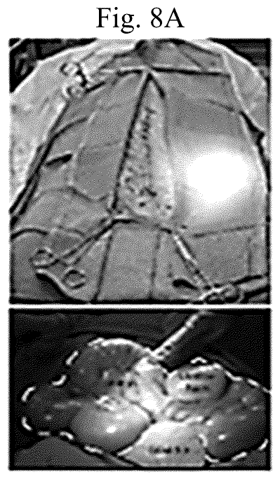

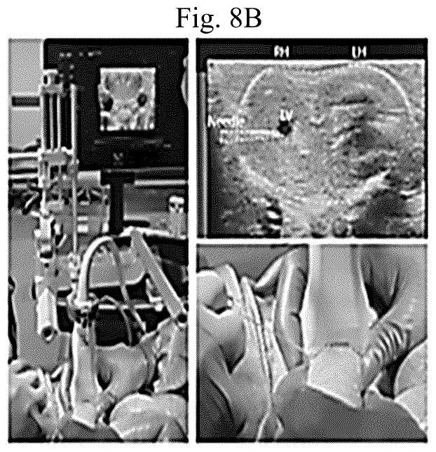

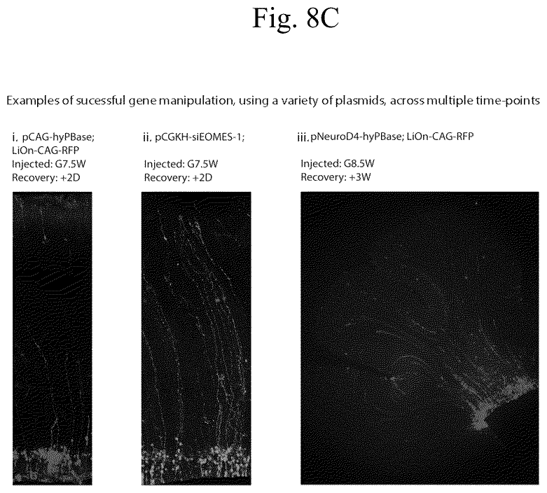

In utero electroporation (IUE). All procedures were performed under sterile conditions. Under anesthesia, a vertical incision was performed mid abdomen, first through the skin and underlying abdominal muscle to expose the uterus (FIG. 8A). Next, the uterus was exteriorized and starting with the uterine horn closest to the cervix, an high resolution ultrasound imaging system (Vevo 3100, FUJIFILM VisualSonics, Inc) was used to locate and confirm the fetal heartbeat in order to establish viability of the fetus. Once viability was established, working with the first fetus, the ultrasound system was used to visualize the fetus's body within the uterus and locate the fetal brain. To maintain position of the fetus within the uterus filled with amniotic fluid, we used customized probe holder that allows precise movement and positioning of the ultrasound probe. The skull of the fetus was gently pressed to the probe for visualization and positioning of the fetal brain for the plasmid delivery. Guided by the high resolution ultrasound imaging system, a sterilized needle with microliter syringe (Hamilton Company) containing plasmid (episomal or integrating plasmids) was visualized in real time and inserted through the maternal uterus and fetus's skull, directly into the lateral ventricle of the fetal brain (FIG. 8B). The anode of a tweezertrode (15 mm diameter, Harvard Apparatus) was placed over the fetal head, and five pulses (125 V, 50-ms duration separated by 1-s intervals) were applied with a BTX ECM830 square pulse generator (Harvard Apparatus) for introduction of the delivered plasmid into the neural stem and progenitor cells in the minipig brain. Following electroporation, the fetal heartbeat was confirmed, and the uterus was returned to the abdomen. The cavity was filled with warm 0.9% saline before suturing the maternal midline incision.

Results: Our experiments targeted the cerebral cortex with episomal expression plasmids, which were allowed to express for 24 hours (FIG. 8C). We also confirmed vectors with longer survival times in which the brains were transfected before gyral formation on 7.5 PCW or 8.5 PCW and then allowed to continue in utero development for 3 weeks until after gyral formations had developed (11.5 PCW; FIG. 7C). Providing the methods with the capability of precisely manipulating genes in neural cells of the gyrencephalic fatal brain will revolutionize our ability to develop and test therapies for the fetal period.

Terminology. Terminology used herein is for the purpose of describing particular embodiments only and is not intended to be limiting of the invention. The following definitions are intended to aid the reader in understanding the present disclosure but are not intended to vary or otherwise limit the meaning of such terms unless specifically indicated. Unless expressly stated, the terms used herein are intended to have the plain and ordinary meaning as understood by those of ordinary skill in the art.

while aspects of the present disclosure have been described in conjunction with the specific embodiments thereof that are proposed as examples, alternatives, modifications, and variations to the examples may be made. The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

It should be noted that, as used in the specification and the appended claims, the singular forms “a,” “an,” and “the” include plural referents unless the context clearly dictates otherwise.

As used herein, the term “and/or” includes any and all combinations of one or more of the associated listed items and may be abbreviated as “/”. A and/or B includes A, B, and (A+B).

As used herein in the specification, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−0.2% of the stated value (or range of values), +/−0.5% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges and values subsumed therein.

Any numerical range recited herein is intended to include all sub-ranges and values subsumed therein. Where a range of values is provided, it is to be understood that each intervening value between an upper and lower limit of the range and any other stated or intervening value in that stated range is encompassed within the disclosure. Where the stated range includes upper and lower limits, ranges excluding either of those limits are also included.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-2, 1-3, 1-4, 1-5, 1-6, 1-7, 1-8, 1-9, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, 9-10 as some examples. A range encompasses its endpoints as well as values inside of an endpoint, for example, the range 0-5 includes 0, >0, 1, 2, 3, 4, <5 and 5.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references and does not constitute an admission as to the accuracy of the content of such references.

1. A genetically modified pig fetus, wherein the pig fetus has been modified to express, enhance, attenuate, or not express at least one gene associated with a brain disease, neurological development, disorder, or condition; wherein the fetus is that of a pig, that of a member of the Suidae family, that of a member of the Sus genus, or that of Sus scrofa domesticus, or that of a minipig.

2. The genetically modified pig of claim 1, wherein the age of the fetus is 14, 15, 16, 17, 18, 19, or 20 weeks.

3. The genetically modified pig of claim 1 that has a brain which has undergone, or is undergoing, cortical folding or gyrification.

4. The genetically modified pig fetus of claim 1, wherein the at least one gene comprises a member of the human NOTCH2NL, ARHGAP11B or NeuroD family, or a pig homolog thereof having at least 70, 75, 80, 85, 90, 95, 99, <100, of 100% sequence identity thereto.

5. The genetically modified pig of claim 1, wherein the at least one gene comprises human LIS1, ARX, DCX, RELN, TUBA1A, CBLN2, SOX2, OLIG2, FOXO1, and/or EGR1, or a pig homolog thereof having at least 70, 75, 80, 85, 90, 95, 99, <100, of 100% sequence identity thereto.

6. The genetically modified pig of claim 1 that has been modified to express a human gene or to increase expression of a pig gene that is a homolog or ortholog of a human gene.

7. The genetically modified pig of claim 1 that has been transformed by a vector expressing a human gene or to stabilize or increase expression of a pig gene that is a homolog or ortholog of a human gene.

8. The genetically modified pig of claim 1 that has been modified to attenuate or knock out expression of a human gene or a pig gene analogous to a human gene.

9. The genetically modified pig of claim 1 that has been transformed by a polynucleotide that knocks out expression of a pig gene analogous to a human gene.

10. A method for detecting effects of administering a polynucleotide into a fetal brain of a pig, comprising:
electroporating in utero a target polynucleotide into the brain of a fetal pig in utero,
imaging the brain structures of the fetal minipig after electroporation and quantifying changes in brain development or function resulting from the administered polynucleotide vector, and
comparing the detected changes to a control or control value to assess effects of expression of the target gene in the fetal brain.

11. The method of claim 10, wherein a needle containing at least one episomal or integration-based plasmid comprising the polynucleotide is inserted into the lateral ventricle to deliver the target polynucleotide.

12. The method of claim 10, wherein electroporation is performed under high resolution ultrasound imaging.

13. The method of claim 10, wherein the target polynucleotide is tagged with a fluorescent dye or other detectable agent.

14. The method of claim 10, wherein the imaging is performed non-invasively.

15. The method of claim 10, wherein the imaging is performed ex vivo after removal and/or sectioning of the fetal brain.

16. The method of claim 10, wherein said imaging is performed in utero.

17. The method of claim 10, wherein said imaging is performed post-mortem ex vivo.

18. The method of claim 10, wherein said imaging comprises evaluation how NPCs give rise to apical radial glial cells, (ii) apical intermediate progenitors, (iii) basal intermediate progenitors and/or (iv) basal radial glia, all of which generate neurons that migrate along radial glial fibers to form the six layers of the cortex in an inside-out fashion; or comprises fate mapping of structures in the developing brain.

19. The method of claim 10, further comprising diagnosing a human subject, who may be a neonate or a fetus, after evaluating the comparative changes in brain structure induced by electroporation of the target polynucleotide and/or conducting MRI or ultrasound visualization of the brain of the human fetus or neonate.

20. The method of claim 10, further comprising treating a human subject, who may be a neonate or a fetus, after evaluating the comparative changes in brain structure of the fetal minipig induced by electroporation of the target polynucleotide and/or administering a drug, biologic or other treatment.
